(19) United States
(12) Patent Application Publication
(10) Pub. No.: US 2021/0275067 A1
(43) Pub. Date: Sep. 9, 2021

(54) BLOOD WITHDRAWAL SYSTEM WITH LANCET EJECTION MECHANISM

(71) Applicant: Roche Diabetes Care, Inc., Indianapolis, IN (US)

(72) Inventor: Roland Hans Wiegand, Mannheim (DE)

(21) Appl. No.: 17/327,419

(22) Filed: May 21, 2021

Related U.S. Application Data
(63) Continuation of application No. PCT/EP2019/081647, filed on Nov. 18, 2019.

(30) Foreign Application Priority Data
Nov. 22, 2018 (EP) 18207772.7

(51) Int. Cl. A61B 5/151

(52) U.S. Cl. CPC A61B 5/15117; A61B 5/150412; A61B 5/15144

(57) ABSTRACT

In one embodiment, a blood withdrawal system for withdrawing blood for diagnostic purposes includes a base device configured to hold a structurally distinct lancet. The base device includes an actuatable lancet ejection mechanism for ejecting the lancet from the base device, and the lancet ejection mechanism includes a movable coupling element. The coupling element includes a coupling structure for coupling the lancet with an external component via the coupling element and subsequently activating the lancet ejection mechanism by operating the external component.

### CROSS-REFERENCE TO RELATED APPLICATIONS

This application is a continuation of International Patent Application No. PCT/EP2019/081647, filed Nov. 18, 2019, which claims the benefit of European Patent Application No. 18207772.7, filed Nov. 22, 2018. The contents of these applications are incorporated herein by reference in their entireties.

### BACKGROUND

The subject application generally relates to blood withdrawal and blood analysis. More particularly, but not exclusively, the subject application relates to a blood withdrawal system with a base device configured to hold a structurally distinct lancet. In one form, the base device may include a lancet ejection mechanism. The subject application also relates to methods for ejecting a lancet from a basic device of a blood withdrawal system.

Blood withdrawal systems may include small lancets which are used to generate a wound in a patient's tissue for withdrawing a blood sample of a small quantity. Some devices which may be used in these systems include a housing with a lancet drive including a drive element, which may be a spring. In operation, these blood withdrawal systems may have an initial state in which the spring is tensed. The system may then be positioned on the patient's tissue and the spring is released, thus forcing the lancet into the tissue. For hygienic reasons, the lancet is only used a single time and disposed thereafter.

Blood withdrawal systems for withdrawing small blood samples may be particularly used by patients suffering from diabetes mellitus. In order to reduce the risk of severe consequences, diligent diabetes therapies require at least four analyses per day. In an attempt to reduce the number of lancet exchanging events, blood withdrawal systems have been developed which include lancet magazines. These magazines are capable of housing several lancets, which may be automatically or semi-automatically exchanged. By way of example, a magazine may include five lancets which are arranged in a circular manner. After a lancet has been used, an exchange mechanism is triggered, upon which the used lancet is returned to the magazine, and the magazine is rotated such that a new, unused lancet may be used for the next blood withdrawal event. After all lancets in the magazine have been used, an ejection mechanism may be triggered by the user, and the magazine may be ejected and subsequently exchanged for a new one.

In order to reduce the risk of unintentional injuries, the lancets of blood withdrawal systems may be located within a housing and only protrude therefrom upon activation of the lancet drive for puncturing the finger. After puncturing, the lancets may be subsequently withdrawn back into the housing. Generally speaking then, blood withdrawal systems may include a housing with an exit opening for at least a tip of the lancet. During puncturing, the lancet can may be moved relative to the housing, and along a straight puncturing path predetermined by the lancet drive. The lancet drive may be based on an elastic drive element such as a metal spring. As mentioned above, the spring may be tensed in an initial state and usually kept in this state by a locking element. The locking element can then be released by the user, for example by pushing a button, upon which a puncturing movement of the lancet drive occurs, which moves the lancet with high speed along the predetermined puncturing path until at least the tip of the lancet protrudes from the housing through the exit opening. In some forms, tensioning of the spring and subsequently triggering the lancet movement are done via a single user operation. If the blood withdrawal system is correctly positioned on the tissue of a patient, a wound is generated by the lancet and the lancet is subsequently withdrawn. The lancet or the lancet magazine may thereafter be removed from the blood withdrawal system by a lancet ejection mechanism and replaced by a new lancet or by a new lancet magazine.

Alternatively, the blood withdrawal systems may be produced as single use devices. Such systems are fully disposable and thus no lancet ejection mechanism is typically necessary. However, single use blood withdrawal systems may be associated with high overall cost for the user in the long term, and they may also produce a significant amount of waste, and in particular plastic waste. Consequently, blood withdrawal systems in which only the lancet is disposed and replaced by a new one may be preferred. In particular, such devices may already include an analytical device, such as a sensor, for testing the blood sample either directly or via insertion of a sensor strip.

As described above, some blood withdrawal systems involve disposal of a lancet which is replaced by a new one. These systems may require an additional lancet ejection mechanism. In this respect, it is irrelevant whether a single lancet or a lancet magazine is employed. The additional lancet ejection mechanism however may have a deleterious effect on the overall cost of the system. In addition, such additional ejection mechanisms are typically realized by implementing a specific slider or button as part of a user interface. In some aspects, the button has to be coupled to the lancet, which complicates the setup of the blood withdrawal system.

Furthermore, any additional part represents a potential source for malfunction of the blood withdrawal system. This may especially be the case for mass produced devices, which have to be manufactured in a simple and cheap manner. Besides the fact that this can lead to less robust blood withdrawal systems, such malfunctions may have severe consequences for the user. If for example the additional lancet ejection mechanism fails, the user may attempt to remove the lancet manually and potentially injure themselves in doing so.

In view of the foregoing, there remains a need for further contributions in this area of technology.

The claimed subject matter is not limited to embodiments that solve any disadvantages or that operate only in environments such as those described above. Rather, this background is only provided to illustrate examples of where the present disclosure may be utilized.

### BRIEF SUMMARY

The present disclosure generally relates to relates to blood withdrawal and blood analysis. More particularly, but not exclusively, the subject application relates to a blood withdrawal system with a base device configured to hold a structurally distinct lancet.

In a first embodiment, a blood withdrawal system for withdrawing blood for diagnostic purposes includes a base device configured to hold a structurally distinct lancet and including an actuatable lancet ejection mechanism for ejecting the lancet from the base device. The lancet ejection mechanism includes a movable coupling element, and the movable coupling element includes a coupling structure configured for coupling the lancet with an external component via the coupling element and subsequently activating the lancet ejection mechanism by operating the external component.

In another embodiment, a kit includes a blood withdrawal system according to the first embodiment and a test strip or an ejection pin.

In yet another embodiment, a method for ejecting a lancet from a basic device of a blood withdrawal system includes coupling an external component with the lancet via a movable coupling element; activating a lancet ejection mechanism by operating the external component; and ejecting the lancet.

In alternative embodiments, assemblies, methods, systems, apparatuses, and devices relating to puncturing skin and blood withdrawal are provided.

This Summary is provided to introduce a selection of concepts in a simplified form that are further described below in the Detailed Description. This Summary is not intended to identify key features or essential characteristics of the disclosed subject matter, nor is it intended to be used as an aid in determining the scope of the disclosed subject matter.

Additional features and advantages will be set forth in the description which follows, and in part will be obvious from the description, or may be learned by the practice of the invention. The features and advantages may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

FIG. 1 shows a schematic, cross-sectional view of a blood withdrawal system which may be used for diagnostic purposes.

FIG. 2 shows a schematic, cross-sectional view of an alternative embodiment of a blood withdrawal system which may be used for diagnostic purposes.

FIG. 3 shows a schematic, cross-sectional view of another alternative embodiment blood withdrawal system which may be used for diagnostic purposes.

FIG. 4 shows a schematic, cross-sectional view of another alternative embodiment blood withdrawal system which may be used for diagnostic purposes.

FIG. 5 shows a schematic, cross-sectional view of another alternative embodiment blood withdrawal system which may be used for diagnostic purposes.

FIG. 6 shows a schematic, cross-sectional view of another alternative embodiment blood withdrawal system which may be used for diagnostic purposes.

### DETAILED DESCRIPTION

For purposes of promoting an understanding of the present disclosure, reference will now be made to the following embodiments and specific language will be used to describe the same. It will nevertheless be understood that no limitation of the scope of the disclosure is thereby intended, such alterations and further modifications in the described subject matter, and such further applications of the disclosed principles as described herein being contemplated as would normally occur to one skilled in the art to which the disclosure relates.

The terms and words used in the following description and claims are not limited to the bibliographical meanings, but, are merely used to enable a clear and consistent understanding of the disclosure. It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

The subject application generally relates to blood withdrawal and blood analysis. More particularly, but not exclusively, the subject application relates to blood withdrawal systems with a base device configured to hold a structurally distinct lancet. In one aspect, the blood withdrawal systems disclosed herein may be used in connection with the monitoring of blood glucose levels.

In one form, a blood withdrawal system for diagnostic purposes includes a base device configured to hold a structurally distinct lancet. The base device includes an actuatable lancet ejection mechanism for ejecting the lancet from the base device, and the lancet ejection mechanism includes a movable coupling element. Furthermore, the movable coupling element includes a coupling structure configured for coupling the lancet with an external component via the coupling element and subsequently activating the lancet ejection mechanism by operating the external component.

As used herein, an external component refers to a component which is not part of and is distinct from the blood withdrawal system. The external component may for example be operated by a user. For example, the external component may be user activated and/or user controlled. Additionally, the movable coupling element may be accessible by the external component. An additional slider, button or additional input means may be avoided by virtue of the movable coupling element including a coupling structure for coupling the lancet with the external component via the coupling element. As the coupling element is configured to activate the lancet ejection mechanism, the ejection of the lancet can be achieved with a blood withdrawal system that requires only a minimum number of components, providing both simplified use and manufacture of the system.

A lancet as described herein may include a sharp blade or needle and is used to generate a puncture in the tissue of a patient. Furthermore, the lancet may include a lancet holder, which may for example be a plastic component at least partially surrounding the lancet for securing the lancet within the basic device of the blood withdrawal system.

Ejection of a lancet may for example be achieved by pushing the lancet out of the basic device or simply by turning the system such that the lancet drops out of the basic device solely due to gravitational force.

The movable coupling element may be a coupling element, which may for example be rotated, sheared, and/or linearly moved within the blood withdrawal system. In one aspect, the coupling element may be made from a single part. For example, the coupling element may be an injection-molded single element. As an example, the coupling element may be a rocker.

In some embodiments, the base device incudes a lancet receptacle for holding the lancet. The lancet receptacle may be releasably connected to the lancet by way of friction and/or force locking. In one form, the lancet may at least be connected to the lancet receptacle until the piercing event has taken place. Thus, in some embodiments, the lancet receptacle is movable in the piercing direction, such that the lancet receptacle guides the lancet during a puncturing event. After the puncturing event, the connection between the lancet receptacle and the lancet may be released.

In one aspect, a blood withdrawal system may further include a drive mechanism, and the drive mechanism may be spring operated. The drive mechanism may be configured to move the lancet along a predetermined path into a protruding position such that a wound can be generated in the skin of the patient. Furthermore, the drive mechanism may also be configured to withdraw the lancet after wound generation.

In some aspects, a blood withdrawal system may further include a structurally distinct lancet. In one embodiment, the basic device of the blood withdrawal system may include a housing, and the housing may enclose an ejection mechanism. The housing may also include a receiving aperture configured to receive a portion of the external component. The receiving aperture may for example be an opening for the external component, such as a slot, cavity, groove, recess, hole, gap, etc., just to provide a few examples. The aperture may extend toward the coupling element and, in one non-limiting form, it may directly extend to the coupling element such that the coupling element is directly accessible by the external component. Thus, the coupling element may be configured to be coupled with the external component. In particular, the movable coupling element may be configured to at least partially transfer a force of the external component acting on the coupling element to the lancet. By way of example, after the coupling element receives a portion of the external component, the remaining portion of the external component may be operated by the user for activating the lancet ejection mechanism. For example, the user may exert a linear force on the external component, i.e., the user may push the external component towards the coupling element, or the user may rotate the external component.

In some embodiments, the housing may further include a lancet opening through which the lancet can protrude for penetrating the skin or through which the lancet can be ejected for disposal.

In some forms, the coupling element may include a pivot to redirect an input linear or rotational movement and/or to transform an input linear movement of the external component into a rotational or sheared movement of the coupling element. As a result, the design of the coupling element may be simplified, and thus the whole blood withdrawal system may possess a simple and easy to manufacture design while also reducing the potential for any malfunctions.

In some embodiments, the pivot may be configured to enable a rotational or sheared movement of at least a portion of the coupling element. By way of example, the pivot may be a hinge joint, film hinge, swivel joint, a flexible structure or any other suitable joint, just to provide a few possibilities.

In some forms, the coupling element of the blood withdrawal system may include two arms which are connected with each other via the pivot in an arrangement providing an angle therebetween. The angle may, for example, be 180°, although other variations are possible. In this case, the coupling element may be a straight bar, such as a rocker for example. A bar of this configuration may be rotatable around the pivot. The coupling element may also be V-shaped, i.e. the angle between the arms may be between 0 and 180°. In some more specific but non-limiting forms, the angle may be 90°, 120°, or 135°. When the angle is 90° for example, the coupling element may be L-shaped. In some forms, the coupling element may include more than two arms. For example, a first arm may be connected to a second arm in a first angle and the second arm may be connected to a third arm in a second angle.

In embodiments in which the coupling element includes two or more arms, one arm may operatively couple and respectively interact with the lancet in its initial configuration prior to ejection. For example, the arm may be pressed against the lancet such that a force is exerted on the lancet and/or engage with an opening or aperture of the lancet.

In some embodiments, the blood withdrawal system may further include a biasing element. The biasing element exerts an initial force on the lancet in its initial configuration, and the initial force is reduced or removed upon activation of the lancet ejection mechanism. The biasing element may be a spring made of metal or an elastic polymer, such as a rubber or silicone block. In certain embodiments, the biasing element may operatively couple and interact with one arm of the coupling element in a manner that results in the arm of the coupling element being pushed towards the lancet. As a result, a force may be exerted on the lancet such that the lancet is clamped between the coupling element and the housing or any other counterpart of the basic device. Therefore, the initial force of the biasing element exerted on the lancet does not necessarily need to be exerted directly on the lancet. In other embodiments, the biasing element may be in direct contact with the lancet and thus directly exert an initial force on the lancet. In such embodiments, activation of the lancet ejection mechanism includes the release of the interaction between the coupling element and the lancet upon which the initial force of the biasing element exerted on the lancet is reduced as the lancet is pushed out of the basic device.

In one embodiment, the coupling element includes a coupling structure for coupling the lancet with an external component, and the external component may be, for example, a test strip or an ejection pin. If the external component is a test strip for example, the ejection of the lancet by coupling the test strip with the lancet via the coupling element and subsequent activation of the lancet ejection mechanism by operating the external component may be performed concomitantly with an analysis of the test strip by an analytical device in the blood withdrawal system. As a result, such a blood withdrawal system is essentially no more complex than a single use blood withdrawal system. In particular, the receiving aperture may concomitantly be used for coupling the test strip with the lancet and subsequently activating the lancet ejection mechanism as well as for analyzing the blood on the test strip. In some embodiments, the coupling structure may be designed to couple with an external component, in particular a pin-shaped or elongated component, of a different type that may serve as an ejection pin, such as a toothpick, a match or a piece of wire, e.g., an end section of a paper clip. It should be appreciated that the test strip or ejection pin must be sufficiently stiff for activating the lancet ejection mechanism. In one aspect, test strips which may be suitable for the purpose described herein may, for example, be cuboid plastic strips with a thickness of 0.5 to 2 mm.

In one aspect, a kit includes a blood withdrawal system as described herein where the coupling element includes a coupling structure for coupling the lancet with an external component, and the external component is a test strip or an ejection pin. The kit also includes a test strip or an ejection pin.

In another aspect, a method for ejecting a lancet from a basic device of a blood withdrawal system includes the steps of coupling an external component with the lancet via a movable coupling element; activating a lancet ejection mechanism by operating the external component; and ejecting the lancet. In some aspects, the external component may be a test strip or an ejection pin, such as a toothpick, a match, or any other element suitable for that purpose.

In some embodiments, the coupling element transforms an input force into an output force, which acts on the lancet or on a biasing element. The biasing element may be a spring or a flexible polymer.

In a further embodiment, a first arm of the coupling element may be moved in a first direction and a second arm of the coupling element may be moved in a second direction. For example, the first and the second direction may be the same, i.e. the pathways of movement may be parallel, or both may be clockwise rotations. Alternatively, the first and second directions may be different from each other, i.e., the pathways of movement are different. It should be appreciated that, as used herein, moving in a direction includes both linear movements, for example a linear movement towards the coupling element, as well as rotational movements, for example in a clockwise or counterclockwise direction.

In another embodiment the coupling element reduces or removes an initial force exerted by a biasing element on the lancet in its initial configuration upon activation of the lancet ejection mechanism. A method utilizing such an embodiment may include coupling an external component with the lancet via a movable coupling element; activating a lancet ejection mechanism by operating the external component; reducing or removing an initial force exerted by a biasing element on the lancet in its initial configuration; and ejecting the lancet.

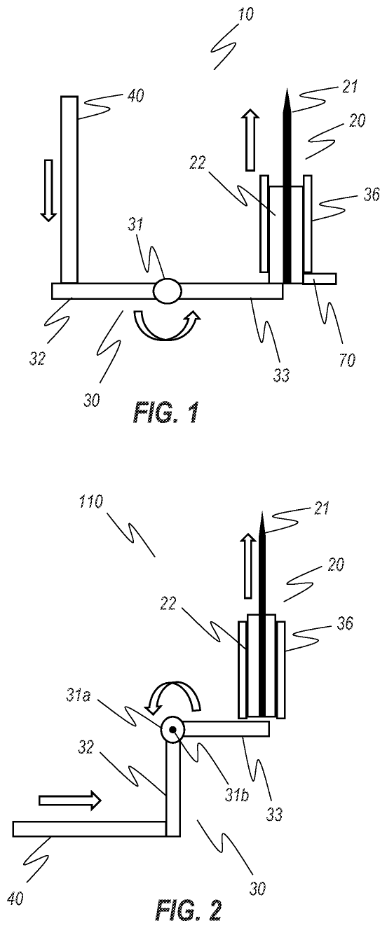

Referring now to FIG. 1, there is illustrated a blood withdrawal system 10 which may be used for diagnostic purposes. The blood withdrawal system 10 includes a base device configured to hold a structurally distinct lancet 20 which includes a blade 21 and a lancet holder 22. The base device additionally includes a lancet receptacle 36 for holding the lancet 20. Upon operating the blood withdrawal system, the lancet receptacle 36 is operated by drive mechanism 70 (not shown in detail) and guides the lancet 20 in a piercing direction into a protruding position. The drive mechanism 70 may be a spring mechanism by way of example. After puncturing the skin of the patient, the drive mechanism 70 may also be configured to withdraw the lancet 20 and/or the lancet receptacle 36. Furthermore, the base device includes an actuatable lancet ejection mechanism for removing the lancet 20 from the base device. The lancet ejection mechanism further includes a movable coupling element 30. The coupling element 30 includes a coupling structure configured for coupling the lancet 20 with an external component 40 via the coupling element 30 and subsequently activating the lancet ejection mechanism by operating the external component 40. The coupling element 30 may, for example, be a rocker.

In the illustrated form, the lancet 20 may be ejected by operating the external component 40 and exerting a force by the coupling element 30 on the lancet 20. The coupling element 30 includes a pivot 31, which may be a joint, such as a hinge joint, film hinge, swivel joint, a flexible structure or any other suitable joint. When the external component 40 is coupled with the lancet 20 by the coupling element 30 and operated, an input linear movement of the external component 40 can be transformed into a rotatory movement of the coupling element 30 around pivot 31, thereby exerting a force on the lancet 20 such that the lancet 20 is ejected from the basic device and the lancet receptacle 36.

Furthermore, as indicated by the straight arrows in FIG. 1, an input linear movement of the external component 40 in a first direction is redirected into a linear movement of the lancet 20 in an opposite, second direction. The coupling element 30 further includes two arms, first arm 32 and second arm 33, which are connected with each other via the pivot 31 in an arrangement providing an angle therebetween of 180°. As can be seen, at an angle of 180°, the coupling element 30 may have the shape of a straight bar. In one form, such a bar may be made from a single piece. In the embodiment shown, the second arm 33 contacts the lancet 20 in its initial configuration prior to ejection.

FIG. 2 illustrates another embodiment of a blood withdrawal system 110 which may be used for diagnostic purposes, where like numerals refer to like features previously described. The blood withdrawal system 110 includes a base device configured to hold a structurally distinct lancet 20. The base device includes a movable coupling element 30, and the movable coupling element 30 includes a first arm 32 and a second arm 33. The first arm 32 and the second arm 33 are connected via pivot 31 in an arrangement providing an angle of 90° therebetween. The pivot 31 includes a female part 31a and a male counterpart 31b.

The pivot 31 is configured to redirect an input linear movement of the external component 40 by 90° to a linear movement of the lancet 20 and to transform the input linear movement of the external component 40 into a rotational movement of the coupling element 30. Upon coupling the external component 40 with the lancet 20 via coupling element 30 and activating the lancet ejection mechanism, the coupling element 30 may conduct a counterclockwise rotation around the male counterpart 31b, thereby ejecting the lancet 20 from the basic device via rotatory movement of the second arm 33. In FIG. 2, no drive mechanism is shown for the sake of clarity. However, a drive mechanism similar to the drive mechanism 70 of FIG. 1 may be present in the blood withdrawal system 110.

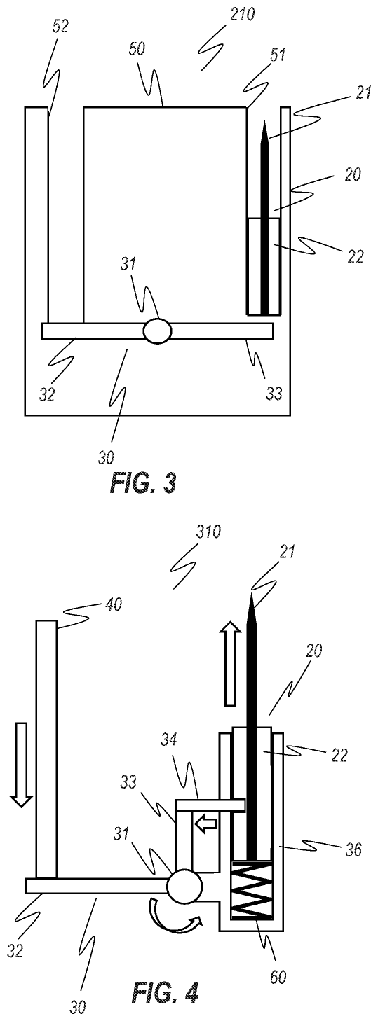

Another embodiment of a blood withdrawal system 210 is depicted in FIG. 3, where like numerals refer to like features previously described. Besides the movable coupling element 30 and the structurally distinct lancet 20, FIG. 3 illustrates a housing 50 of the basic device enclosing the ejection mechanism. The housing 50 includes a receiving aperture 52 which extends towards the coupling element 30 and which is configured to receive a portion of an external component. In the embodiment shown, the receiving aperture 52 is designed as an additional opening in the housing 50. The receiving aperture 52 may be configured to receive the external component to enable coupling of the external component with the lancet 20 via the coupling element 30 and to subsequently activate the lancet ejection mechanism by operating the external component. Even though the coupling element 30 in the embodiment of FIG. 3 is designed similar to the coupling element of FIG. 1, it should be appreciated that any other coupling element described herein may also be employed together with a receiving aperture.

As can be seen, the lancet 20, including the blade 21, is fully in its initial configuration, i.e., in a retracted position in which the structurally distinct lancet 20 is shielded by the housing 50. The initial configuration may be assumed either when the blood withdrawal system 210 has not yet been used or when the blood withdrawal system 201 has just been used by activation of a lancet drive (not shown), which guides the lancet 20 through a lancet opening 51 in the housing and into the tissue of a patient. After the wound has been generated, the lancet 20 may be retracted by the lancet drive for safety reasons. The patient may now contact a test strip with the blood exiting the wound.

In some embodiments, the blood withdrawal system 210 includes an analytic device, such as a sensor, for analyzing the blood obtained from the wound. The patient may insert a test strip, i.e. an external component, into the receiving aperture 52, and the test strip may be used for analyzing the blood and may also be coupled with the lancet 20 via the coupling element 30. The test strip may subsequently activate the lancet ejection mechanism by operating the external component in a manner similar to that described above in connection with FIG. 1 which entails the ejection of the lancet 20 from the basic device.

FIG. 4 shows another embodiment of a blood withdrawal system 310, where like numerals refer to like features previously described. The blood withdrawal system 310 includes the structurally distinct lancet 20 which includes the blade 21 and the lancet holder 22. The blood withdrawal system 310 also includes a base device with an actuatable lancet ejection mechanism with the coupling element 30. As can be seen, the coupling element 30 includes the pivot 31, around which the coupling element 30 is rotatable or at which the coupling element 30 may be sheared. The coupling element 30 additionally includes first arm 32, second arm 33, and a third arm 34. The angle between the first arm 32 and the second arm 33 at the pivot is 90°. The angle between the second arm 33 and the third arm 34 is also 90°. Optionally, the coupling element 30 may also include a second pivot between the second arm 33 and the third arm 34. In the particular embodiment shown, the pivot 31 is a film hinge. The third arm 34 is engaged with the lancet 20 in its initial configuration prior to ejection.

As indicated in FIG. 4, the engagement is achieved by a recess within lancet holder 22, which is configured to receive at least a portion of the third arm 34. The blood withdrawal system 310 further includes a lancet receptacle 36 and a biasing element 60 in the form of a spring is positioned in the lancet receptacle 36 and acts as a biasing element which exerts an initial force on the lancet 20 in its initial configuration. In this embodiment, the biasing element 60 is in direct contact with the lancet 20. When the blood withdrawal system 310 is used for generating a wound in the tissue of a patient, the lancet 20, the lancet receptacle 36 as well as the coupling element 30 may be moved in the puncturing direction (indicated by the vertical, upwards arrow) via a drive mechanism. The movement may be triggered by a drive mechanism (not shown), similar to the drive mechanism 70 of FIG. 1.

It is further shown in FIG. 4 that the lancet receptacle 36 may be connected to the pivot 31. Similarly, the lancet receptacle 36 may be configured to carry the pivot 31. Furthermore, in FIG. 4 the initial configuration is illustrated in which the biasing element 60 is tensed and blocked from releasing by the engagement of the third arm 34 with the lancet 20. When the external component 40, such as an ejector pin or a test strip, is coupled with the lancet 20 via the coupling element 30, it may exert a force on the first arm 32, which leads to a rotational and/or sheared movement of the coupling element 30 around or at the pivot 31, thereby enabling retraction of the third arm 34 out of the recess within the lancet holder 22 (as indicated by the arrows). In this manner, the third arm 34 is removed from engagement with the lancet 20 and the energy stored in the biasing element 60 can be released and the initial force exerted on the lancet 20 is reduced. As a result, the lancet 20 may be pushed out of the basic device.

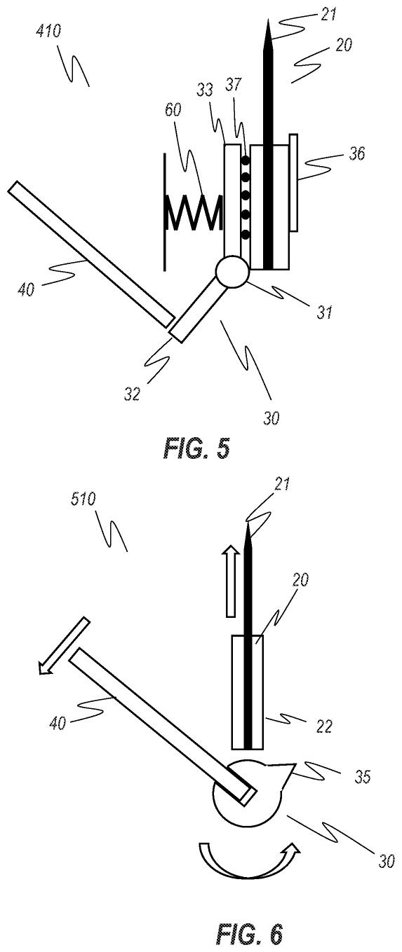

Another embodiment of a blood withdrawal system 410 with the biasing element 60 is shown in FIG. 5, where like numerals refer to like features previously described. The biasing element 60 may be an elastic or resilient element, such as a coil spring or a block made from resilient material, such as a rubber or silicone block. The biasing element 60 exerts a force on the lancet 20. In contrast to the embodiment shown in FIG. 4, the biasing element 60 is not in direct contact with the lancet 20. The blood withdrawal system 410 further includes a linear bearing 37, which may be a linear ball bearing or linear friction bearing for example. The linear bearing 37 may be part of the coupling element 30 or the lancet receptacle 36, or it may be an additional part of the base device. The linear bearing 37 is configured to enable linear movement of the lancet 20 and optionally the lancet receptacle 36 in the puncturing direction to bring the lancet in a protruding position. This may for example be achieved by allowing a sliding movement of the lancet 20 in the puncturing direction.

The coupling element 30 further includes first arm 32, which is connected with second arm 33 at the pivot 31 in an arrangement providing an angle of approximately 165° therebetween. The movable coupling element 30 includes a coupling structure for coupling the lancet 20 with an external component 40 via the coupling element 30 and for subsequently activating the lancet ejection mechanism. When the external component 40 exerts a force on the first arm 32, counterclockwise rotatory movement of the coupling element 30 results. As a result, the second arm 33 compresses the biasing element 60, thereby releasing the lancet 20 from its initial configuration. If the user wants to remove the lancet 20, the device may be turned such that the lancet may be released therefrom due to gravitational force.

Another embodiment of a blood withdrawal system 510 is shown in FIG. 6, where like numerals refer to like features previously described. The blood withdrawal system 510 includes a base device and the structurally distinct lancet 20. The base device includes an actuatable lancet ejection mechanism for ejecting the lancet from the base device with a movable coupling element 30. The coupling element 30 has an essentially circular cross-section with a nose 35 and a recess or slot for receiving an external component 40. In one form, the coupling element 30 may generally be disk-shaped with the exception of the nose 35 and the slot or recess.

As indicated by the arrows, the coupling element 30 may be rotationally movable. More specifically, in the illustrated form, the coupling element 30 has a pivot (not shown) to transform an input movement of the external component 40 into a linear movement of the lancet 20. If the user inserts the external component 40 into the recess, the coupling element 30 is rotationally movable by turning the external component 40. For example, the coupling element is configured such that the nose 35 is in close proximity or already in contact with the lancet 20. Therefore, operation of the external component 40 by the user (i.e., by rotating the external component 40) activates the lancet ejection mechanism. In particular, a force is exerted by the external component 40 on the coupling element 30 such that the lancet 20 is ultimately ejected from the base device.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

### REFERENCE NUMERAL LISTING

10 blood withdrawal system
20 lancet
21 blade
22 lancet holder
30 coupling element
31 pivot
32 first arm
33 second arm
34 third arm
35 nose
36 lancet receptacle
37 linear bearing
40 external component
50 housing
51 lancet opening
52 receiving aperture
60 biasing element
70 drive mechanism

What is claimed is:

1. A blood withdrawal system for withdrawing blood for diagnostic purposes, comprising:
a base device configured to hold a structurally distinct lancet and including an actuatable lancet ejection mechanism for ejecting the lancet from the base device;
wherein the lancet ejection mechanism includes a movable coupling element; and
wherein the movable coupling element includes a coupling structure for coupling the lancet with an external component via the coupling element and subsequently activating the lancet ejection mechanism by operating the external component.

2. The blood withdrawal system of claim 1, further comprising the structurally distinct lancet.

3. The blood withdrawal system of claim 1, wherein the base device comprises a lancet receptacle for holding the lancet.

4. The blood withdrawal system of claim 1, wherein the base device comprises a housing enclosing the ejection mechanism, and the housing further comprises a receiving aperture configured to receive a portion of the external component.

5. The blood withdrawal system of claim 1, wherein the coupling element comprises a pivot configured for at least one of redirecting an input linear or rotational movement of the external component and transforming an input linear movement of the external component into a rotational or sheared movement of the coupling element.

6. The blood withdrawal system of claim 5, wherein the coupling element comprises two arms connected with each other via the pivot in an arrangement providing an angle therebetween.

7. The blood withdrawal system of claim 6, wherein one of the arms operatively couples with the lancet in an initial configuration thereof prior to ejection.

8. The blood withdrawal system of claim 7, further comprising a biasing element, wherein the biasing element is configured to exert an initial force on the lancet in the initial configuration thereof, and wherein the initial force is reduced or removed upon activation of the lancet ejection mechanism.

9. The blood withdrawal system of claim 1, further comprising a biasing element, wherein the biasing element is configured to exert an initial force on the lancet in an initial configuration thereof, and wherein the initial force is reduced or removed upon activation of the lancet ejection mechanism.

10. The blood withdrawal system of claim 1, wherein the coupling element comprises a coupling structure for coupling the lancet with an external component.

11. The blood withdrawal system of claim 10, wherein the external component is a test strip or an ejection pin.

12. A kit, comprising the blood withdrawal system according to claim 11 and a test strip or an ejection pin.

13. A method for ejecting a lancet from a basic device of a blood withdrawal system, comprising:
coupling an external component with the lancet via a movable coupling element;
activating a lancet ejection mechanism by operating the external component; and
ejecting the lancet.

14. The method of claim 13, wherein the coupling element transforms an input force into an output force which acts on the lancet or on a biasing element.

15. The method of claim 14, wherein the coupling element includes a first arm and a second arm, the first arm of the coupling element is moved in a first direction, and the second arm of the coupling element is moved in a second direction.

16. The method claim 15, wherein the coupling element reduces or removes an initial force exerted by a biasing element on the lancet in an initial configuration upon activation of the lancet ejection mechanism.

17. The method of claim 13, wherein the coupling element includes a first arm and a second arm, the first arm of the coupling element is moved in a first direction, and the second arm of the coupling element is moved in a second direction.

18. The method claim 13, wherein the coupling element reduces or removes an initial force exerted by a biasing element on the lancet in an initial configuration upon activation of the lancet ejection mechanism.
